(19) United States
(12) Patent Application Publication
(10) Pub. No.: US 2026/0041661 A1
(43) Pub. Date: Feb. 12, 2026

(54) THREE-COMPONENT PRODRUG, PHARMACEUTICAL COMPOSITION THEREOF, AND MEDICAL USE THEREOF

(71) Applicant: J2H BIOTECH INC., Gyeonggi-do (KR)

(72) Inventors: Hyung-Chul RYU, Gyeonggi-do (KR); Jae-Sun KIM, Gyeonggi-do (KR); Jee-Woong LIM, Gyeonggi-do (KR); Ju Young LEE, Gyeonggi-do (KR); Kwanghyun CHOI, Gyeonggi-do (KR); Duk-Ho CHANG, Gyeonggi-do (KR); Seung-Yong LEE, Gyeonggi-do (KR)

(73) Assignee: J2H BIOTECH INC., Gyeonggi-do (KR)

(21) Appl. No.: 19/100,902

(22) PCT Filed: Aug. 2, 2022
(86) PCT No.: PCT/KR2022/011411

§ 371 Date: Feb. 3, 2025

(51) Int. Cl. A61K 31/235; A61P 25/28; A61P 37/06; C07C 69/604

(52) U.S. Cl. CPC A61K 31/235; A61P 25/28; A61P 37/06; C07C 69/604

(57) ABSTRACT

The present disclosure relates to a novel compound that allows for the production of monomethyl fumarate after being administered, a pharmaceutical composition containing same as an active ingredient, and a medicinal use thereof.

### CROSS REFERENCE TO RELATED APPLICATIONS

This application is a National Stage of International Application No. PCT/KR2022/011411, filed Aug. 2, 2022. The International Application was published in Korea on Feb. 8, 2024, as International Publication No. WO2024/029639 A1. The disclosure of the above identified application is incorporated herein by reference in its entirety.

### TECHNICAL FIELD

The present disclosure relates to a novel precursor drug of monomethyl fumarate known to be useful for the treatment or improvement of various diseases such as psoriasis, multiple sclerosis, atopy, asthma, arthritis, inflammatory bowel disease, lupus, amyotrophic lateral sclerosis, Huntington disease, Alzheimer's disease, Parkinson's disease, macular degeneration, sleep apnea, radiologically isolated syndrome, scleroderma, neuropathic pain, chronic pain, gout, diabetic complications including diabetic foot ulcers, cerebrovascular disease, cardiovascular disease, cancer, and tumors. The present disclosure also relates to a pharmaceutical composition comprising such novel precursor drug as an active ingredient. The present disclosure also relates to medical uses utilizing such a precursor drug.

### BACKGROUND ART

Dimethyl fumarate (DMF) is a methyl ester compound of fumaric acid and is the active ingredient in the commercially available Fumaderm (for psoriasis) and Tecfidera (for multiple sclerosis). These products are oral pharmaceuticals. Dimethyl fumarate is rapidly metabolized to monomethyl fumarate (MMF) after oral administration, and the substance that actually exhibits the medicinal effect is known as monomethyl fumarate. In other words, dimethyl fumarate can be said to be a precursor drug of the active metabolite monomethyl fumarate.

The physiological mechanism of action of monomethyl fumarate has been studied in various aspects to date, and several research results have been reported that elucidate its immunomodulatory function, antioxidant effect, protection of nerve cells, and anti-inflammatory effect. (Journal of Neuroinflammation, 2012, 9, 163; Redox Biology, 2015, 169; Critical Reviews in Immunology, 2013, 33(4), 307; Journal of Immunology, 2011, 187(10), 5015; Perspectives in Medicinal Chemistry, 2015, 7, 1; Psoriasis:Targets and Therapy, 2015, 5, 9) In the case of the already commercialized Fumaderm product, it was developed as a complex of four fumaric acid esters (FAEs), including dimethyl fumarate, calcium ethyl hydrogen fumarate, magnesium ethyl hydrogen fumarate, and zinc ethyl hydrogen fumarate. This drug has been shown to be effective in patients with moderate psoriasis, and its mechanism is reported to be related to selective immunomodulation (induction of Th2-selective cytokine secretion). (British Journal of Dermatology, 1999, 141, 424.)

Tecfidera, a product containing dimethyl fumarate as the main ingredient, is a multiple sclerosis treatment. The active metabolite, monomethyl fumarate, has been reported to exhibit therapeutic effects on multiple sclerosis primarily through activation of the Nrf2 (Nuclear factor (erythroid-derived 2)-like 2) pathway. (Brain, 2011, 678; Human Molecular Genetics, 2017, 26(15), 2864.) In addition, antioxidant Nrf2 activators such as monomethyl fumarate exhibit therapeutic effects on various neurodegenerative diseases such as Alzheimer's disease, Huntington's disease, Parkinson's disease, and amyotrophic lateral sclerosis, including multiple sclerosis. (British Journal of Pharmacology, 2017, 174(12), 1750.)

Monomethyl fumarate or dimethyl fumarate is known to be useful in the treatment, improvement, or prevention of atopy, macular degeneration, sleep apnea, radiologically isolated syndrome associated with multiple sclerosis, scleroderma, systemic sclerosis-associated pulmonary arterial hypertension, cancer, and tumors, in addition to the above diseases. (https://clinicaltrials.gov/ct2/show/NCT02438137, https://clinicaltrials.gov/ct2/show/NCT02739542, https://clinicaltrials.gov/ct2/show/NCT02981082, J Neuroinflammation. 2015; 12: 239., Aging (Albany NY). 2016 July; 8(7): 1289-1290, J Invest Dermatol. 2018 January; 138(1):78-88. doi: 10.1016/j.jid.2017.08.024. Epub 2017 Sep. 1, Nature Scientific Reports|7:41605|DOI: 10.1038/srep41605, Front Immunol. 2018; 9: 1896. J Investig Allergol Clin Immunol 2018; Vol. 28(3): 182-215).

Although the above-mentioned psoriasis, multiple sclerosis, inflammatory diseases, or neurodegenerative diseases are all intractable diseases that are very difficult or impossible to treat, dimethyl fumarate (DMF) products are recognized as having excellent clinical efficacy in the treatment of some diseases such as psoriasis and multiple sclerosis.

Due to the nature of the above diseases, long-term administration of dimethyl fumarate preparations for at least one month is inevitable, but dimethyl fumarate preparations are known to have severe side effects.

For example, Tecfidera, used as a treatment for multiple sclerosis, is commercialized in capsule formulations (120 mg and 240 mg), and is administered as an initial dose of 120 mg twice a day for 7 days, and then increased to the recommended dose of 240 mg twice a day. However, side effects due to long-term use, such as gastrointestinal disorders (diarrhea, nausea, abdominal pain, upper abdominal pain), flushing, lymphopenia, and progressive multifocal leukoencephalopathy, commonly occur. In particular, gastrointestinal disorders and flushing are very common side effects, occurring in more than 20% and 40%, respectively, of patients. In addition, due to the risk of the above side effects, it is recommended to take Tecfidera with food to improve tolerance, and in cases of severe flushing or gastrointestinal disorders, the dosage may be temporarily reduced to 120 mg twice a day, once. However, even in such cases, the dosage must be increased to the recommended dosage of 240 mg twice a day, once within 1 month.

To complement these shortcomings of dimethyl fumarate, precursor drugs of monomethyl fumarate (MMF) with a novel chemical structure have been developed. Among them, ALKS8700 (Alkemes, USA) developed diroximel fumarate and received new drug approval from the FDA in 2019 (product name: VUMERITY, U.S. Pat. No. 8,669,281). In addition, XP23829 (Xenoport, USA) has currently completed phase 2 clinical trials (U.S. Pat. No. 8,148,414). These candidates are all precursor drugs of monomethyl fumarate for development as a treatment for multiple sclerosis, and they focus on improving the serious side effects of the dimethyl fumarate, especially gastrointestinal disorders. Among them, candidate XP23829 has not sufficiently demonstrated the special feature of bioequivalence (bioequivalent therapy) compared to toxicity when used as a control drug with existing dimethyl fumarate preparations in terms of efficacy for treating multiple sclerosis. The dose administered in phase 2 clinical trials of XP23829 was designed as a high-dose regimen compared to existing dimethyl fumarate preparations, at 400 mg and 800 mg once or twice daily, respectively.

Meanwhile, diroximel fumarate (DRF), which received new drug approval from the FDA in 2019, reported that the incidence and severity of gastrointestinal side effects were improved compared to dimethyl fumarate (DMF) in phase 3 clinical trials. Nevertheless, gastrointestinal side effects (gastrointestinal (GI) treatment-emergent adverse events (TEAEs)) were observed in more than 30% of all administered patients. This result is a 10% decrease compared to the 40% incidence of gastrointestinal side effects that occurred in the patient group administered dimethyl fumarate (DMF) in phase 3 clinical trials. However, it was reported that 14.9% of subjects in the EVOLVE-MS-1 phase 3 clinical program discontinued DRF administration, and 6.3% of them discontinued due to drug-induced side effects. [Reference: Improving the Gastrointestinal Tolerability of Fumaric Acid Esters: Early Findings on Gastrointestinal Events with Diroximel Fumarate in Patients with Relapsing-Remitting Multiple Sclerosis from the Phase 3, Open-Label EVOLVE-MS-1 Study: Adv Ther, Jul. 17, 2019 (https://doi.org/10.1007/s12325-019-01085-3)]

In particular, diroximel fumarate did not significantly improve flushing, one of the most common side effects of dimethyl fumarate. EVOLVE-MS-2 is a phase 3 clinical trial in which 506 patients with relapsing-remitting multiple sclerosis (RRMS) were treated with DRF (462 mg twice daily) and DMF (240 mg twice daily) for 5 weeks, respectively, and the frequency and severity of side effects were compared. The most commonly reported side effects in both treatment groups were flushing, diarrhea, and nausea, in that order, which were reported in 32.8%, 15.4%, and 14.6% of the DRF group and in 40.6%, 22.3%, and 20.7% of the DMF group, respectively. Gastrointestinal side effects and flushing side effects are reported to be the two most important reasons for discontinuing DMF treatment. In particular, in the above clinical phase 3, flushing is a side effect that appears at a very low rate in the placebo group, so it has the characteristic of appearing in more than 30% of patients administered DMF or DRF. [Reference: https://www.medscape.com/viewarticle/916496]

### DISCLOSURE

### Technical Problem

Therefore, the purpose of the present disclosure is to provide a precursor drug of monomethyl fumarate that reduces side effects such as gastrointestinal side effects and flushing and exhibits excellent physiological activity and pharmacokinetics, a pharmaceutical composition comprising the drug, and medical uses of the drug for treating or improving various diseases such as immune system disorders, neurodegenerative diseases, and inflammatory diseases.

### Technical Solution

In order to achieve the above purpose, the present disclosure provides a compound represented by the following chemical formula 1:

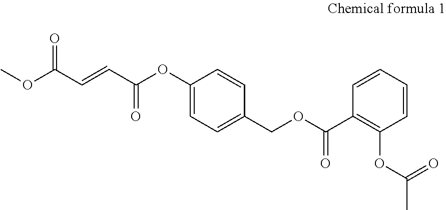 [SMILES: COC(=O)/C=C/C(=O)Oc1ccc(COC(=O)c2ccccc2OC(C)=O)cc1]

The inventors of the present invention have prepared and evaluated various precursor drugs. Among the various compounds evaluated, the compound above showed excellent effects in terms of improving side effects and pharmacokinetics, and in addition to these aspects, it was confirmed that it showed excellent properties or characteristics as a precursor drug, thereby completing the present invention.

That is, the precursor drug composed of three components characterized by having a specific structure in which monomethyl fumarate and 2-acetoxybenzoic acid are linked by 4-hydroxybenzyl alcohol (HBA) was confirmed to be suitable for the purpose of the present invention in various aspects, thereby completing the present invention.

As used herein, the phrase “compound(s) of this/the invention” includes any compound(s) of Chemical Formula 1, as well as clathrates, hydrates, solvates, or polymorphs thereof.

As used herein, the term “polymorph” refers to solid crystalline forms of a compound of this disclosure or complex thereof. Each polymorph of a compound with the same chemical structure exhibits different physical, chemical and/or spectroscopic properties. Different physical properties include, but are not limited to stability (e.g., to heat or light), compressibility and density (important in formulation and product manufacturing), and dissolution rates (which can affect bioavailability). Differences in stability can result from changes in chemical reactivity (e.g., differential oxidation, such that a dosage form discolors more rapidly when comprised of one polymorph than when comprised of another polymorph) or mechanical characteristics (e.g., tablets crumble on storage as a kinetically favored polymorph converts to thermodynamically more stable polymorph) or both (e.g., tablets of one polymorph are more susceptible to breakdown at high humidity). Different physical properties of polymorphs can affect their processing. For example, one polymorph might be more likely to form solvates or might be more difficult to filter or wash free of impurities than another due to, for example, the shape or size distribution of particles of it.

As used herein, the term “solvate” means a compound or its salt according to this disclosure that further includes a stoichiometric or non-stoichiometric amount of a solvent bound by non-covalent intermolecular forces. Preferred solvents are volatile, non-toxic, and acceptable for administration to humans in trace amounts.

As used herein, the term “hydrate” means a compound or its salt according to this disclosure that further includes a stoichiometric or non-stoichiometric amount of water bound by non-covalent intermolecular forces.

As used herein, the term “clathrate” means a compound or its salt in the form of a crystal lattice that contains spaces (e.g., channels) that have a guest molecule (e.g., a solvent or water) trapped within.

The compound represented by the above chemical formula 1 is metabolized in the body after oral administration as shown in the following scheme 1:

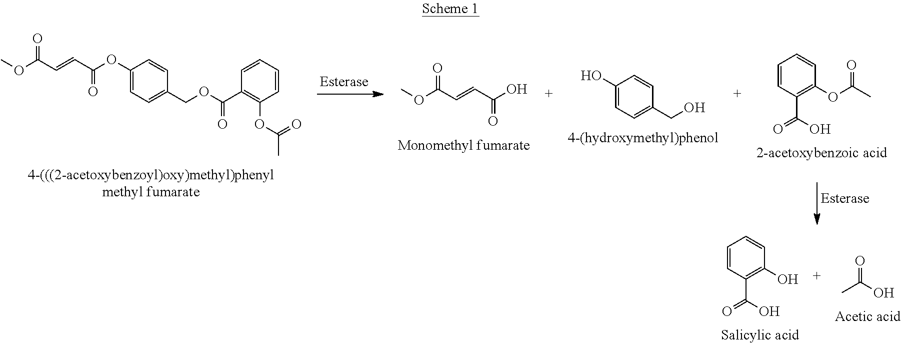

The compound represented by the above chemical formula 1 is metabolized by esterase in the body to produce MMF (monomethyl fumarate), HBA (4-(hydroxymethyl) phenol), and 2-acetoxybenzoic acid, respectively. In addition, 2-acetoxybenzoic acid (aspirin) is also metabolized by esterase in the body to salicylic acid and acetic acid.

The compound of the above chemical formula 1 (3-component prodrug) exhibits not only the upregulation effect of NRF2/HO-1, which is the mechanism of action of MMF; the antioxidant effect in astrocytes, which is the physiological activity of HBA; and the anti-inflammatory and analgesic effect of 2-acetoxybenzoic acid and its metabolite, salicylic acid, but also the flushing relief effect can be simultaneously expressed as described below.

The present inventors designed, synthesized, and evaluated various 3-component prodrugs. For example, the compound represented by the following chemical formula 2 has HBA as a linker, the same as chemical formula 1, but the positions of MMF and 2-acetoxybenzoic acid are different from those of the compound of chemical formula 1.

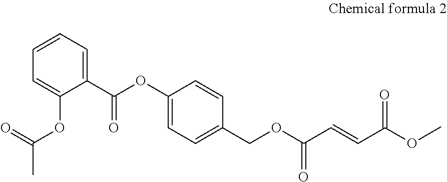 [SMILES: COC(=O)/C=C/C(=O)OCc1ccc(OC(=O)c2ccccc2OC(C)=O)cc1]

The compound of the above chemical formula 2 was confirmed to not detect sufficient amounts of MMF, 2-acetoxybenzoic acid, and its metabolite salicylic acid in in vivo pharmacokinetic tests using mice, rats, and dogs. In other words, although it has a very similar structure to chemical formula 1, there was a significant difference in its metabolites.

Since both compounds of chemical formula 1 and 2 have four ester bonds, they can be competitively hydrolyzed by esterase in vivo, but the inventors have confirmed that the metabolites differ depending on the slight difference in chemical structure. For example, the compound of chemical formula 2 was confirmed to be relatively stable in vivo as a benzyl ester compared to the compound of chemical formula 1, and the hydrolysis of the methyl ester of the MMF functional group appears competitively and more quickly. This is shown in the above reaction scheme 2:

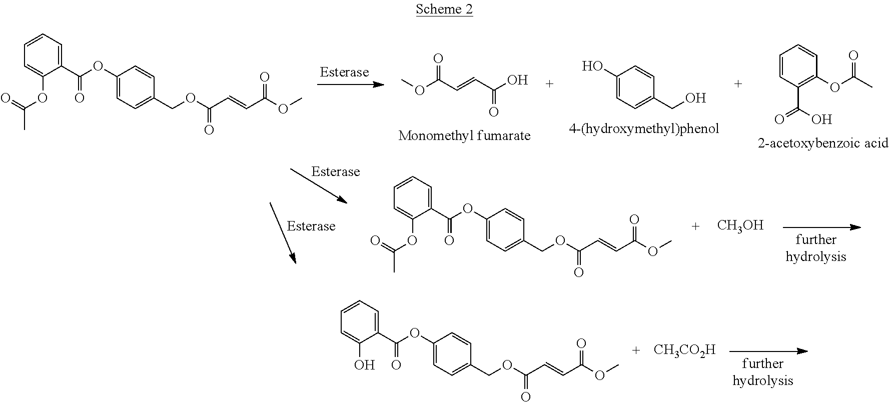

In the second pathway of the above scheme 2, the methyl ester of MMF is hydrolyzed before or more competitively than the ester bond of the carrier HBA, and as a result, it is expected that the blood concentration of MMF sufficient to meet the physiological activity requirement through body metabolism cannot be achieved.

In addition, the inventors designed various derivatives of the above chemical formulas 1 and 2 and conducted synthesis and evaluation.

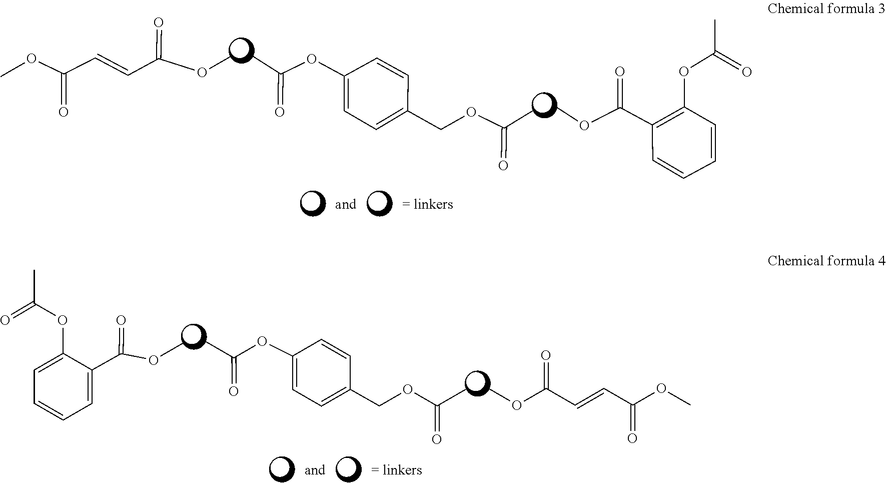

In the above chemical formulas 3 and 4, the linker may be considered to have various combinations, such as alkyl having 1 to 4 carbon atoms; alkyl substituted with a halogen, methyl, dimethyl, hydroxy, amino group, etc.; aryl; aryl substituted with a halogen, alkyl, hydroxy, amino group, etc.; heteroaryl; heteroaryl substituted with a halogen, alkyl, hydroxy, amino group, etc.; alkenyl having 2 to 4 carbon atoms; alkynyl having 2 to 4 carbon atoms; alkenyl or alkynyl substituted with a halogen, methyl, dimethyl, hydroxy, amino group, etc.

The present inventors evaluated the oral absorption rate and blood metabolites in rats for compounds of the above chemical formulas 3 and 4 that had excellent physicochemical properties and storage stability. Many of the compounds of the above chemical formulas 3 and 4 had low oral absorption rates as their molecular weight and fat solubility increased. In addition, in many cases, hydrolysis of the ester functional group preceded under the pH conditions of the stomach, and even when oral absorption occurred, various undesirable metabolites were formed due to the competitive hydrolysis reaction of the six esters. As a result, it was not possible to confirm a precursor drug that was hydrolyzed in vivo in a complete form as in the above chemical formula 1.

Accordingly, the present inventors confirmed the three-component prodrug of the above chemical formula 1 as the most complete form and completed the present invention.

The compound represented by the above chemical formula 1 of the present invention can be synthesized, for example, by the following route:

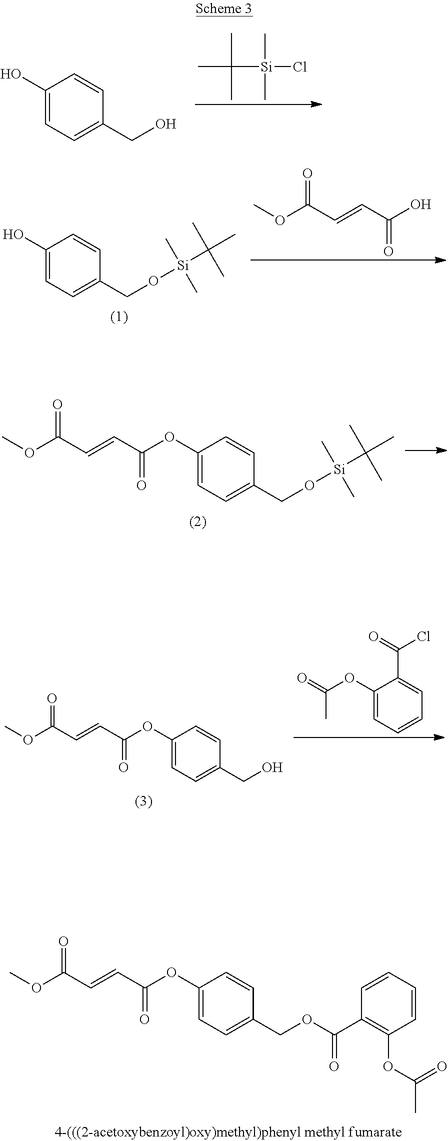

This synthesis method is one example, and the final product of the above scheme 3 represented by the above chemical formula 1 can be manufactured by various methods by those skilled in the art.

The inventors of the present invention have modified the chemical structure of the monomethyl fumarate compound to create a novel compound having a new structure that has sufficient oral bioavailability for the expression of medicinal effects upon oral administration and desirable pharmacokinetic profiles, physicochemical properties, etc. After orally administering the novel compound, the concentration of monomethyl fumarate exposed in the blood is ultimately proportional to the efficacy of the drug for treating or improving various diseases. Therefore, by comparing the concentration of monomethyl fumarate exposed in the blood after intravenous administration of monomethyl fumarate and the concentration of monomethyl fumarate exposed in the blood through a metabolic process after orally administering the novel compound according to the present invention, the doses of intravenous administration and oral administration can be proportionally applied.

Meanwhile, another component of the three-component prodrug according to the present invention is 2-acetoxybenzoic acid, which is the same as the component sold under the product name Aspirin. 2-acetoxybenzoic acid is an anti-inflammatory analgesic with anti-inflammatory and antioxidant effects. It has been reported as a clinical result that when multiple sclerosis patients take aspirin, their exercise capacity is improved and body temperature increase due to exercise is reduced. [Reference: Aspirin is an effective pretreatment for exercise in multiple sclerosis: A double-blind randomized controlled pilot trial, Multiple Sclerosis Journal, Volume: 24 issue: 11, page(s): 1511-1513] Meanwhile, it has been reported that aspirin can improve multiple sclerosis through the protective effect of Regulatory T cells in an experimental autoimmune encephalomyelitis, EAE mice model. [Reference: Aspirin ameliorates experimental autoimmune encephalomyelitis through interleukin-11-mediated protection of regulatory T cells, Science Signaling 27 Nov. 2018: Vol. 11, Issue 558,] In addition, aspirin is known to alleviate the flushing side effect caused by DMF, and both DMF and DRF are recommended to be used together with aspirin to alleviate the flushing side effect.

The other component of the three-component prodrug of the present invention is 4-hydroxybenzyl alcohol (HBA) used as a linker. HBA is a natural substance present in medicinal plants, etc., and is also called Gastrodigenin, and is also known as an indicator component of, for example, Gastrodia elata. HBA penetrates the blood-brain barrier and has been reported to have physiological activities such as improving brain damage in rats by antioxidant action. [Reference: 4-hydroxybenzyl alcohol ameliorates cerebral injury in rats by antioxidant action. Neurochem Res. 2011 February; 36(2):339-46.] In addition, HBA has been reported to have the effect of preventing brain damage and behavioral disorders by activating Nrf2 and PDI (protein disulphide isomerase) in a rat model of cerebral ischemia. [Reference: p-hydroxybenzyl alcohol prevents brain injury and behavioral impairment by activating Nrf2, PDI, and neurotrophic factor genes in a rat model of brain ischemia, Molecules and Cells, March 2011, Volume 31, Issue 3, pp 209-215]

Another embodiment of the present disclosure provides a pharmaceutical composition comprising a compound of the above formula 1 and a pharmaceutically acceptable carrier.

The pharmaceutically acceptable carrier may be, for example, a carrier for oral administration or a carrier for parenteral administration. The carrier for oral administration may include lactose, starch, cellulose derivatives, magnesium stearate, stearic acid, and the like. In addition, the carrier for parenteral administration may include water, a suitable oil, saline, aqueous glucose, glycol, and the like. The pharmaceutical composition may further include a stabilizer and a preservative. Suitable stabilizers include antioxidants such as sodium bisulfite, sodium sulfite, or ascorbic acid. Suitable preservatives include benzalkonium chloride, methyl-or propyl-paraben, and chlorobutanol. For other pharmaceutically acceptable carriers, reference may be made to well-known literature in the field to which the present invention pertains.

One embodiment of the present disclosure also provides a pharmaceutical composition for treating or improving psoriasis, multiple sclerosis, atopy, asthma, arthritis, inflammatory bowel disease, lupus, amyotrophic lateral sclerosis, Huntington disease, Alzheimer's disease, Parkinson's disease, macular degeneration, sleep apnea, radiologically isolated syndrome, scleroderma, neuropathic pain, chronic pain, gout, diabetic complications including diabetic foot ulcers, cerebrovascular disease, cardiovascular disease, cancer or tumor, comprising a compound of formula 1 according to the present invention as an active ingredient. That is, the present invention provides medical uses of the compound of chemical formula 1 according to the present invention for treating or improving those diseases. The compound of the present invention can maximize the efficacy of MMF and minimize its side effects. Examples of the cerebrovascular disease or cardiovascular disease include arteriosclerosis, pulmonary hypertension, diabetic cardiomyopathy, stroke, etc.

Another embodiment of the present disclosure also provides a method of treating or ameliorating psoriasis, atopy, macular degeneration, multiple sclerosis, asthma, arthritis, inflammatory bowel disease, lupus, amyotrophic lateral sclerosis, Huntington's disease, Alzheimer's disease, Parkinson's disease, sleep apnea, radiologically isolated syndrome, scleroderma, neuropathic pain, chronic pain, gout, diabetic complications including diabetic foot ulcers, cerebrovascular disease, cardiovascular disease, cancer or tumor, comprising administering to a subject in need thereof a therapeutically effective amount of a compound of the chemical formula 1. In another embodiment, the subject is a human. In one embodiment, the treatment is a preventative treatment. In another embodiment, the treatment is a palliative treatment. In yet another embodiment, the treatment is a restorative treatment.

As used herein, “effective amount or effective dose” refers to an amount of a compound of the present invention sufficient to delay or minimize the occurrence of the aforementioned disease or a symptom thereof: or to provide a therapeutic benefit in the treatment or management of such disease.

The pharmaceutical composition of the present invention can be administered to mammals including humans by any route of administration, and can be administered orally or parenterally. However, the oral route is more preferable in that the compounds of the present invention show excellent oral absorption rates.

The parenteral administration method may include, but is not limited to, intravenous, intramuscular, intraarterial, intramedullary, intrathecal, intracardiac, transdermal, subcutaneous, intraperitoneal, intranasal, intestinal, topical, sublingual, or rectal administration. For example, the pharmaceutical composition of the present invention may be prepared as an injectable formulation and administered by lightly pricking the skin with a 30-gauge thin injection needle, or by directly applying it to the skin.

The pharmaceutical composition of the present invention may be formulated as a preparation for oral or parenteral administration according to the above-described route of administration.

In the case of preparations for oral administration, the composition of the present invention can be formulated into powders, granules, tablets, pills, dragees, capsules, liquids, gels, syrups, slurries, suspensions, etc. using methods known in the art. For example, oral preparations can be obtained by mixing an active ingredient with a solid excipient, grinding the mixture, adding a suitable auxiliary agent, and then processing the mixture into a granule to obtain a tablet. Examples of suitable excipients may include fillers such as sugars including lactose, dextrose, sucrose, sorbitol, mannitol, xylitol, erythritol, maltitol, etc.; starches including corn starch, wheat starch, rice starch, potato starch, etc.; and celluloses including cellulose, methyl cellulose, sodium carboxymethyl cellulose, hydroxypropylmethylcellulose, etc. The pharmaceutical formulation of the present disclosure may also use a binder such as polyvinylpyrrolidone, hydroxypropylmethylcellulose, and the like, and in some cases, disintegrating agents such as cross-linked polyvinylpyrrolidone, agar, alginic acid, or sodium alginate may also be added. Furthermore, the pharmaceutical composition of the present invention may further include an anticoagulant, a lubricant, a wetting agent, a fragrance, an emulsifier, a preservative, and the like. In addition, in order to relieve gastrointestinal irritation, an enteric coating or microencapsulation may be used so that the drug is released when passing through the intestines rather than being released in the stomach.

In the case of a preparation for parenteral administration, it may be formulated in the form of an injection, a cream, a lotion, an ointment for external use, an oil, a moisturizer, a gel, an aerosol, and a nasal inhaler by a method known in the art.

The total dosage of the pharmaceutical composition of the present invention can be administered to a patient as a single dose, or can be administered by a fractionated treatment protocol in which multiple doses are administered for a long period of time. The pharmaceutical composition of the present invention can vary the content of the active ingredient depending on the symptoms of the disease. Preferably, the preferred total dosage of the active ingredient of the present invention can be about 0.01 μg to 1,000 mg per 1 kg of patient body weight per day, and most preferably 1 mg to 100 mg. However, the dosage of the active ingredient of the present invention can be determined by a person having ordinary knowledge in the art by considering various factors such as the administration route and the number of treatments as well as the patient's age, weight, health condition, sex, severity of the disease, diet, excretion rate, etc. The pharmaceutical composition according to the present invention is not particularly limited in its formulation, administration route, and administration method as long as it exhibits the effects of the present invention.

In addition, the pharmaceutical composition of the present invention can be administered as an individual therapeutic agent or in combination with other therapeutic agents. When administered in combination with other therapeutic agents, the composition of the present invention and the other therapeutic agents may be administered simultaneously, separately, or sequentially. In this case, the other therapeutic agents may be substances already known to have therapeutic or ameliorating effects on various diseases such as immune system disorders, neurodegenerative diseases, and inflammatory diseases. When the pharmaceutical composition of the present invention is administered in combination with other therapeutic agents, the composition of the present invention and the other therapeutic agents may be formulated separately in separate containers, or may be combined in the same formulation.

An exemplary formulation for administering the compound presented in the present invention to the human body includes an enteric-coated tablet. Such tablets may be manufactured using a method well known in the field to which the present invention pertains, and may be specifically manufactured with ingredients as shown in Table 1 below.

TABLE 1

| (Unit: weight %) | | Composition 1 | Composition 2 |
|---|---|---|---|
| Tablet before | Active ingredients (chemical | 10 | 12 |
| coating | formula 1 compound) | | |
| (Uncoated tablet) | Mannitol | 55 | 48 |
| | Hydroxypropyl cellulose | 20 | 25 |
| | Sodium stearyl fumarate | 1 | 1 |
| Membrane for | Polyvinylpyrrolidone | 2 | 2 |
| separation | | | |
| Enteric coating | Hypromellose phthalate | 10.3 | 10.3 |
| | Dibutyl sebacate | 1.2 | 1.2 |
| | Titanium dioxide | 0.5 | 0.5 |

### Advantageous Effects

The present invention provides the precursor compound effective for treating or improving various diseases such as immune system disorders, neurodegenerative diseases, and inflammatory diseases, a pharmaceutical composition comprising it as an active ingredient, their medical uses, and therapeutic methods comprising administering it to a subject in need of treatment or prevention. The compound according to the present invention is an active ingredient of a pharmaceutical product, and not only has various advantages in various aspects such as physicochemical properties, but is also excellent in bioavailability, pharmacokinetics, etc. in that three components are exposed to the body through biodegradation after oral administration. In addition, the compound according to the present invention exhibits even more excellent effects in terms of side effects.

### BRIEF DESCRIPTION OF FIGURES

FIG. 1 shows the results of evaluating the CD4 T cell and CD8 T cell infiltration rates in the lymph nodes of each test group in a multiple sclerosis disease model using an autoimmune encephalomyelitis-induced mouse (EAE mouse, experimental autoimmune encephalomyelitis).

FIG. 2 shows the results of evaluating the CD4 T cell and CD8 T cell infiltration rates in the central nervous system of each test group in a multiple sclerosis disease model using an autoimmune encephalomyelitis-induced mouse (EAE mouse, experimental autoimmune encephalomyelitis).

FIG. 3 shows the results of evaluating the Th1 cell and Th17 cell infiltration rates in the lymph nodes of each test group in a multiple sclerosis disease model using an autoimmune encephalomyelitis-induced mouse (EAE mouse, experimental autoimmune encephalomyelitis).

FIG. 4 shows the results of measuring the skin flushing side effect due to drug administration, and the results of analyzing the concentration of prostaglandin D2 (PGD2) in the blood in a mouse model, which is a standard test method.

### MODE FOR INVENTION

The present invention will be described in more detail based on the following examples, but this is not intended to limit the scope of the present invention. In addition, those skilled in the art will be able to make various modifications and alterations to the present invention within a scope that does not harm the spirit of the present invention.

### Example 1: Synthesis of the Compound of Chemical Formula 1

Step 1: Synthesis of 4-((tert-butyldimethylsilyloxy)methyl)phenol (1)

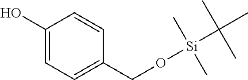 [SMILES: CC(C)(C)[Si](C)(C)OCc1ccc(O)cc1]

1.0 g of 4-Hydroxybenzyl alcohol was dissolved in 5 ml of dimethylformamide and 1.15 g (0.95 equivalents) of tert-butylmethylsilyl chloride was additionally dissolved. The mixture was stirred while lowering the temperature using an ice bath. After about 30 minutes, a mixed solution of 2.5 ml of dimethylformamide and 1.12 ml (1 equivalent) of triethylamine was slowly added dropwise under low temperature. After stirring at room temperature for 16 hours, the organic layer was extracted using 30 ml of water and 20 ml of methyl tert-butyl ether. The extracted organic layer was washed with 10 ml of brine and 10 ml of ammonium chloride aqueous solution, respectively. Then, it was dried over anhydrous magnesium sulfate and filtered, and the filtrate was concentrated under reduced pressure to obtain a brown oily compound.

¹H NMR (400 MHz, DMSO-d6) δ9.271 (s, 1H), 7.80 (d, 2H), 6.70 (d, 2H), 4.558 (s, 2H), 0.868 (s, 9H), 0.038 (s, 6H)

Properties: Brown Oil/Yield ≥90%

Step 2: Synthesis of 4-((tert-butyldimethylsilyloxy)methyl)phenyl Methyl Fumarate (2)

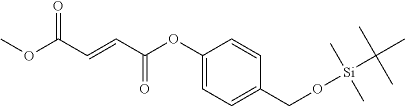 [SMILES: COC(=O)/C=C/C(=O)Oc1ccc(CO[Si](C)(C)C(C)(C)C)cc1]

4-((tert-butyldimethylsilyloxy)methyl)phenol (1) 1 g was added to 10 ml of dichloromethane solution, and additionally 0.54 g (1 equivalent) of monomethyl fumarate and 0.025 g (0.05 equivalent) of 4-dimethylaminopyridine were added and stirred at room temperature to form a suspension. After about 30 minutes, 1.6 g (2 equivalents) of 1-ethyl-3-(3-dimethylaminopropyl)carbodiimide hydrochloride was added and stirred. After the suspension changed to a clear solution, dichloromethane was removed through reduced pressure distillation. Thereafter, 20 ml of ethyl acetate and 20 ml of water were used for extraction, and the obtained organic layer was dried over anhydrous magnesium sulfate, filtered, and concentrated under reduced pressure to obtain a mixture. The obtained mixture was separated and purified through a silica gel column (ethyl acetate:heptane=1:4), then concentrated under reduced pressure to obtain a brown emulsion compound.

¹H NMR (400 MHz, DMSO-d6) δ7.328 (d, 2H), 7.144 (d, 2H), 6.936 (s, 2H), 4.677 (s, 2H), 3.742 (s, 3H), 0.863 (s, 9H), 0.043 (s, 6H) Properties: Brown Oil/Yield 65%

Step 3: Synthesis of 4-(hydroxymethyl)phenyl Methyl Fumarate (3)

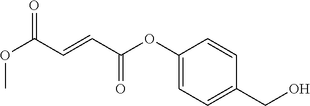 [SMILES: COC(=O)/C=C/C(=O)Oc1ccc(CO)cc1]

1 g of 4-((tert-butyldimethylsilyloxy) methyl) phenyl methyl fumarate (2) was added to a mixture of 5 ml of dimethyl sulfoxide and 1 ml of water, and the mixture was refluxed at 150° C. Thin layer chromatography was used to confirm whether compound (2) was completely consumed. When compound (2) was completely consumed, the organic layer was separated by extraction using 30 ml of water, 6 ml of ethyl acetate, and 12 ml of methyl tert-butyl ether. After that, the mixture was washed with 10 ml of brine and 10 ml of ammonium chloride aqueous solution, respectively, dried over anhydrous magnesium sulfate, filtered, and the filtrate was concentrated under reduced pressure to obtain a white solid. The obtained solid was dissolved in 2 ml of methyl tert-butyl ether, and 20 ml of heptane was added, and the recrystallized solid was obtained by filtering.

¹H NMR (400 MHz, DMSO-d6) δ7.420 (d, 2H), 7.21 (d, 2H), 7.023 (s, 2H), 5.296 (t, 1H), 4.546 (d, 2H), 3.83 (s, 3H)

Properties: White powder/Yield 85%

Step 4: Synthesis of 4-((acetoxybenzoyloxy)methyl)phenyl Methyl Fumarate (Chemical Formula 1)

1 g of 4-(hydroxymethyl)phenyl methyl fumarate (3) was dissolved in 10 ml of dichloromethane solution, 0.65 ml (1.1 equivalents) of triethylamine was added, and the mixture was stirred while lowering the temperature using an ice bath. After about 30 minutes, 0.84 g (1 equivalent) of 2-(chlorocarbonyl)phenyl acetate was dissolved in 10 ml of dichloromethane and slowly added dropwise to the compound (3) aqueous solution. After about 1 hour, the ice bath was removed and stirred at room temperature for 16 hours. Dichloromethane was removed by distillation under reduced pressure, and then extracted using 10 ml of ethyl acetate and 10 ml of water. The extracted organic layer was dried over anhydrous magnesium sulfate and filtered, and the filtrate was concentrated under reduced pressure to obtain a mixture. The obtained mixture was separated and purified through a silica gel column (ethyl acetate: methyl tert-butyl ether: heptane=1:2:7) and concentrated under reduced pressure to obtain a white solid compound.

¹H NMR (400 MHz, DMSO-d6) δ7.992 (d, 1H), 7.691 (t, 1H), 7.54 (d, 2H), 7.423 (t, 2H), 7.227-7.288 (m, 3H), 6.993 (s, 2H), 5.316 (s, 2H), 3.79 (s, 3H), 2.092 (s, 3H)

Properties: White powder/Yield 70%

### Experimental Example 1: Pharmacodynamic Evaluation Using Beagle Dog

The pharmacodynamic test on the compound of Example 1 was conducted as follows. The test substance was the compound of Chemical Formula 1 prepared in Example 1, and the control substance was dimethyl fumarate (Sigma-Aldrich, catalog number 50744), the main ingredient of Tecfidera product. After the test substance and the control substance were administered orally once to beagle dogs, the concentration of monomethyl fumarate released into the blood by the metabolic process was tracked and analyzed over time, thereby proving the efficacy of the compound of the present invention.

Meanwhile, as another control group, dimethyl fumarate and aspirin were administered together, and the blood concentrations of MMF, aspirin, and salicylic acid in the blood were compared with those in the group administered with Chemical Formula 1.

The administration groups in this experiment were as shown in Table 2 below.

TABLE 2

| Administration | | | |
|---|---|---|---|
| group (Number | | | |
| of animals) | Substance | Dosage | Vehicle |
| G1 (n = 3) | Compound of chemical formula 1 | 16.6 mg/kg | 10% NMP in corn oil |
| G2 (n = 3) | Dimethyl fumarate | 6.0 mg/kg | 10% NMP in corn oil |
| G3 (n = 3) | Dimethyl fumarate, | 6.0 mg/kg, | 10% NMP in corn oil |
| | Aspirin (Co-administration) | 7.5 mg/kg | |

Test and control substances were each prepared in the same manner and administered to beagle dogs at a dose of 0.0417 mmol/kg, and blood was collected at a set time and plasma was separated. Drug analysis was performed using HPLC (XBridge column C₁₈, Waters, mobile phase 0.1% formic acid:acetonitrile (30:70, %/%)) and MS/MS (ESI positive, MRM). Beagle dog plasma and each commercial standard solution were mixed in a 9:1 ratio, and concentrations of 5, 50, 100, 500, 1000, and 5,000 ng/ml were prepared and calibrated. In addition, QC samples were prepared by mixing beagle dog plasma and QC standard solutions in a 9:1 ratio, and concentrations of 100, 750, and 2,500 ng/ml were prepared. The preprocessing method was to transfer 100 μl of plasma sample to a centrifuge tube, add 10 μl of internal standard solution and 300 μl of methanol, and mix for about 30 seconds. The tube was centrifuged at 3,000×g (4° C.) for about 5 minutes, and the supernatant was transferred to an LC vial and injected into the device. Then, the concentration of the active ingredient, i.e., monomethyl fumarate, in the beagle dog plasma was quantified using a pre-validated analysis method. The pharmacokinetic parameters were calculated using the WinNonlin 5.2 (Pharsight, USA) program, and AUC₀₋ₜ, AUC_{0-∞}, Cₘₐₓ, Tₘₐₓ, and t_{1/2} were calculated using Noncompartment modeling (best fit). The pharmacokinetic parameter results were expressed as the mean and standard deviation (SD), and statistically processed using the SPSS program (Statistical Package for the Social Sciences, 10.0K, USA).

The test results, the bioavailability after oral administration of the control and test substances are summarized in Tables 3 and 4 below.

### Comparison of Blood MMF Concentrations in Groups G1 and G2

TABLE 3

| | | | | Relative Absorption | |
|---|---|---|---|---|---|
| | | AUC₀₋₂₄ | Cmax | (vs. dimethyl | Route of |
| Classification | | (hr*ng/ml) | (ng/ml) | fumarate, %) | administration |
| Control | Dimethyl | 7,929 ± 1,297 | 1,605 ± 586 | — | Oral |
| substance | fumarate | | | | |
| (G2) | | | | | |
| Test | Example 1 | 9,568 ± 1,519 | 2,274 ± 274 | 120.7% | Oral |
| substance | | | | | |
| (G1) | | | | | |

### Comparison of Blood MMF Concentrations in Groups G1 and G3 (Co-Administration)

TABLE 4

| | | | | Relative | |
|---|---|---|---|---|---|
| | | AUC₀₋₂₄ | Cmax | Absorption (vs. | Route of |
| Classification | | (hr*ng/ml) | (ng/ml) | coadministration, %) | administration |
| Coadministration | Dimethyl | 7,367 ± 2,109 | 2,018 ± 334 | — | Oral |
| (G3) | fumarate + | | | | |
| | Aspirin | | | | |
| Test substance | Example 1 | 9,568 ± 1,519 | 2,274 ± 274 | 129.9% | Oral |
| (G1) | | | | | |

For example, in the case of Example 1 compound (G1), the average AUCₜ was 9,568 hr*ng/ml, the average AUCᵢ was 11,009 hr*ng/ml, the average Cₘₐₓ was 2,274 ng/ml, the average Tₘₐₓ was 1.17 hours, the average t_{1/2} was 7.62 hours, and the relative absorption compared to dimethyl fumarate (G2) was 120.7% based on blood MMF. Meanwhile, in the case of Example 1 compound (G1), the relative absorption of aspirin was 111.4% and the relative absorption of salicylic acid was 108.8% compared to the co-administration of dimethyl fumarate+aspirin (G3) (based on AUC₀₋₂₄). Table 5 shows the blood concentrations (ng/mL) of monomethyl fumarate over time in the oral administration group of Example 1 compound compared to the control group administered dimethyl fumarate, and Table 6 shows the blood concentration trends of monomethyl fumarate, aspirin, and salicylic acid in the oral administration group of Example 1 compound.

TABLE 5

| (ng/mL) | Example 1 Compound | | (ng/mL) | Dimethyl Fumarate | |
|---|---|---|---|---|---|
| time (h) | mean | SD | time (h) | mean | SD |
| 0 | 0 | 0 | 0 | 0 | 0 |
| 0.25 | 1171 | 1103 | 0.25 | 1399 | 937 |
| 0.5 | 1868 | 743 | 0.5 | 1127 | 445 |
| 1 | 1305 | 1183 | 1 | 1124 | 729 |
| 2 | 1246 | 698 | 2 | 987 | 175 |
| 4 | 782 | 121 | 4 | 862 | 182 |
| 6 | 452 | 345 | 6 | 382 | 199 |
| 8 | 389 | 87 | 8 | 194 | 103 |
| 12 | 248 | 94 | 12 | 197 | 32 |
| 24 | 103 | 86 | 24 | 83 | 53 |
| AUC₀₋₁₂ | 9568 | 1519 | AUC₀₋₁₂ | 7929 | 1297 |
| AUC_{inf} | 11009 | 3111 | AUC_{inf} | 9434 | 2555 |
| Cmax | 2274 | 274 | Cmax | 1605 | 586 |
| Tmax | 1.17 | 0.76 | Tmax | 1.50 | 2.17 |
| T1/2 | 7.62 | 3.79 | T1/2 | 8.15 | 5.32 |

TABLE 6

| (ng/mL) | Monomethyl fumarate | | Aspirin | | Salicylic acid | |
|---|---|---|---|---|---|---|
| time (h) | mean | SD | mean | SD | mean | SD |
| 0 | 0 | 0 | 0 | 0 | 0 | 0 |
| 0.25 | 1171 | 1103 | 302 | 160 | 1390 | 586 |
| 0.5 | 1868 | 743 | 460 | 124 | 2396 | 214 |
| 1 | 1305 | 1183 | 380 | 234 | 2551 | 927 |
| 2 | 1246 | 698 | 534 | 88 | 3693 | 507 |
| 4 | 782 | 121 | 577 | 26 | 4263 | 231 |
| 6 | 452 | 345 | 360 | 39 | 2958 | 187 |
| 8 | 389 | 87 | 241 | 11 | 2038 | 301 |
| 12 | 248 | 94 | 123 | 32 | 1212 | 397 |
| 24 | 103 | 86 | 31 | 34 | 195 | 296 |
| AUC₀₋₂₄ | 9568 | 1519 | 4869 | 936 | 36674 | 9386 |
| AUC_{inf} | 11009 | 3111 | 5234 | 1427 | 38771 | 12349 |
| Cmax | 2274 | 274 | 586 | 42 | 4263 | 231 |
| Tmax | 1.17 | 0.76 | 3.33 | 1.15 | 4.00 | 0.00 |
| T1/2 | 7.62 | 3.79 | 5.47 | 3.57 | 2.25 | 1.16 |
| Relative absorption | 129.9% | | 111.4% | | 108.8% | |
| compared to | | | | | | |
| coadministration | | | | | | |

### Experimental Example 2: Evaluation of Efficacy in an Animal Model of Multiple Sclerosis (EAE)

The symptoms and efficacy of the compound of Example 1 according to the present invention were evaluated using experimental autoimmune encephalomyelitis (EAE mice, experimental autoimmune encephalomyelitis).

### (1) Induction of EAE Disease and Drug Administration

Specifically, 6-8 week old female C57BL/6 mice were used for the EAE experiment. EAE induction was performed as follows. Myelin oligodendrocyte glycoprotein 35-55 (MOG) was prepared and mixed with complete Freund's adjuvant (CFA; Difco, USA) containing Mycobacterium Tuberculosis (10 mg/ml H37RA; Difco, USA), and this emulsion was subcutaneously injected into mice. Simultaneously, 250 ng of pertussis toxin (PTX, List Biological Lab, USA) was injected intraperitoneally on day 0 and day 2, respectively. The test substance was suspended in corn oil containing 2.5% DMSO and orally administered twice a day from day 2 to the final day (day 21). (n=10) A summary of the administered substances for each group is summarized in Table 7 below.

TABLE 7

| Administration | | | | | |
|---|---|---|---|---|---|
| Group | Group Type | Test Substance | Dosage | | Vehicle |
| G1 | Normal Group | — | — | | — |
| G2 | Negative | vehicle | — | | DMSO 2.5%, |
| | Control | | | | corn oil 97.5% |
| G3 | Positive | Dimethyl fumarate | 30 | mg/kg | DMSO 2.5%, |
| | Control 1 | | | | corn oil 97.5% |
| G4 | Positive | Dimethyl fumarate | 60 | mg/kg, | DMSO 2.5%, |
| | Control 2 | | | | corn oil 97.5% |
| G5 | Test Group | Compound of chemical | 82.8 | mg/kg | DMSO 2.5%, |
| | (Low dose) | formula 1 | | | corn oil 97.5% |
| G6 | Test Group | Compound of chemical | 165.6 | mg/kg | DMSO 2.5%, |
| | (High dose) | formula 1 | | | corn oil 97.5% |

| | | | | |
|---|---|---|---|---|
| G7 | Positive | Dimethyl fumarate + | 60 mg/kg + | DMSO 2.5%, |
| | Control 3 | Aspirin | 75 mg/kg | corn oil 97.5% |

### (2) Efficacy of Improving Clinical Symptoms

The clinical symptom scale (BAE score) of the BAB animal model induced by MOG peptide was evaluated in 10 stages according to Table 8 below along with daily body weight, and expressed as a clinical score of 0 to 5 points.

TABLE 8

| Clinical | |
|---|---|
| score | Clinical observations |
| 0.0 | No obvious changes in motor function |
| 0.5 | Tail tip drooping or weak |
| 1.0 | Tail entire drooping |
| 1.5 | Tail entire drooping and more than a part of hind legs |
| 2.0 | Tail entire drooping and hind legs weak |
| 2.5 | Tail entire drooping and hind legs dragging |
| 3.0 | Tail entire drooping and hind legs paralyzed |
| 3.5 | Tail entire drooping and hind legs completely paralyzed |
| 4.0 | Tail entire drooping and hind legs completely paralyzed |
| | and front legs partially paralyzed |
| 4.5 | Hind legs completely paralyzed and front legs partially |
| | paralyzed, no movement in cage |
| 5.0 | Death due to paralysis |

The experimental results are shown in Table 9 below.

TABLE 9

| | Clinical | CD4 T | CD8 T | Th1 | Th17 |
|---|---|---|---|---|---|
| | Score | Cell | Cell | Cell | Cell |
| | (Day 21) | (%) | (%) | (%) | (%) |
| Normal Group | 0.0 | 0.8 | 0.9 | 3.5 | 4.6 |
| Vehicle Group | 3.2 | 23.2 | 16.0 | 15.4 | 12.4 |
| DMF 30 mpk | 2.3 | 13.1 | 8.8 | 10.2 | 10.1 |
| DMF 60 mpk | 1.3 | 13.8 | 8.5 | 7.9 | 6.3 |
| Compound of | 2.0 | 11.2 | 7.8 | 8.8 | 8.8 |
| chemical | | | | | |
| formula 1 | | | | | |
| 82.8 mpk | | | | | |
| Compound of | 1.5 | 6.5 | 4.7 | 6.4 | 7.5 |
| chemical | | | | | |
| formula 1 | | | | | |
| 165.6 mpk | | | | | |
| DMF 60 mpk + | 1.1 | 5.8 | 4.1 | 5.4 | 4.8 |
| Aspirin 75 mpk | | | | | |

As a result of the experiment, EAE induction using MOG showed symptoms from the 10^{th} day, and the clinical score of the negative control group (G2) that was not administered the drug was approximately 3, indicating that the disease induction was appropriate. The control substance dimethyl fumarate used in the positive control group significantly reduced the severity of the disease at low concentrations (G3) and high concentrations (G4), respectively.

In the test group administered with the compound of chemical formula 1, both low dose (G5) and high dose (G6) showed significant clinical score improvement effects, and in particular, the clinical score improvement was superior at the high dose than at the low dose, indicating a dose correlation. In addition, the low dose group (G5) of the compound of chemical formula 1 showed a similar level of efficacy to the low dose group (G3) of the dimethyl fumarate group administered at the same molar ratio, and the high dose group (G6) of the compound of chemical formula 1 also showed a similar level of efficacy to the high dose group (G4) of the dimethyl fumarate group administered at the same molar ratio. This was confirmed at a similar level of results in the aspirin combination group (G7).

### (3) Efficacy of Suppressing Immune Cell Infiltration

Multiple sclerosis is known to be caused fundamentally by the attack of specific immune cells on the nervous tissue. For example, increased infiltration rates of CD4 or CD8 expressing T cells and Th1 or Th17 cells in the lymph nodes (LN) or central nervous system (CNS) are known to be very important disease aggravation factors. Accordingly, the present researchers extracted lymph nodes, and brains and spinal cords (CNS) from mice 3 weeks after antigen injection for each test group of the EAE animal model and calculated the ratio (%) of the number of immune cells using a flow cytometry analyzer.

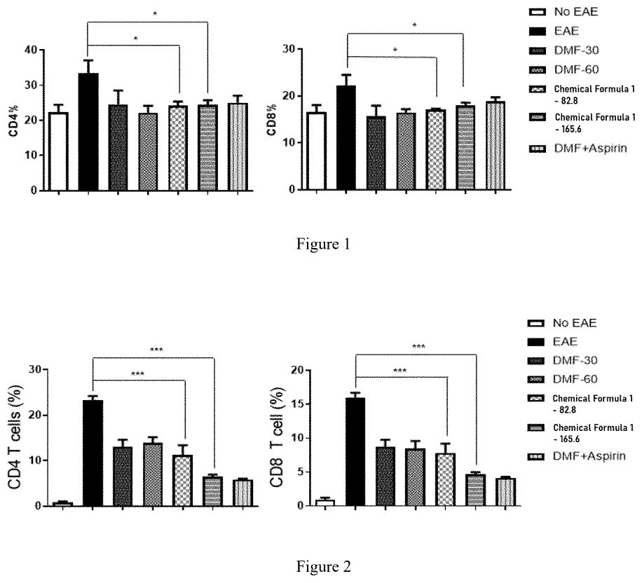

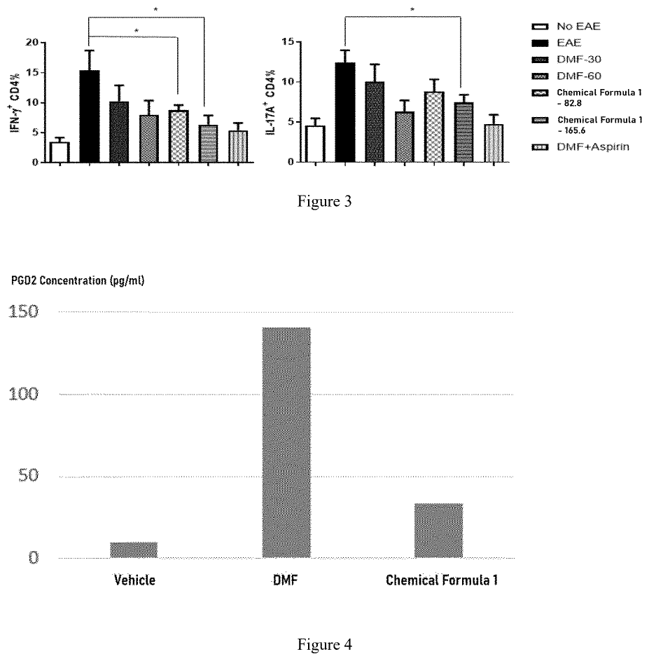

The results are shown in FIG. 1 (lymph node, CD4 T cell and CD8 T cell infiltration rates), FIG. 2 (central nervous system, CD4 T cell and CD8 T cell infiltration rates), and FIG. 3 (lymph node, Th1 cell and Th17 cell infiltration rates).

As shown in FIG. 1, both the low-dose administration group (G5) and the high-dose administration group (G6) of the compound of chemical formula 1 showed a significant decrease in the infiltration rate of CD4 and CD8 T cells in the lymph nodes compared to the disease-induced group (G2).

In addition, as shown in FIG. 2, both the low-dose administration group (G5) and the high-dose administration group (G6) of the compound of chemical formula 1 showed a significant decrease in the infiltration rate of CD4 and CD8 T cells in the central nervous system compared to the disease-induced group (G2).

In addition, as shown in FIG. 3, both the low-dose administration group (G5) and the high-dose administration group (G6) of the compound of chemical formula 1 showed a decrease in the infiltration rate of Th1 and Th17 cells in the lymph nodes compared to the disease-induced group (G2).

### Experimental Example 3: Toxicity Evaluation

We evaluated whether flushing, a common side effect of dimethyl fumarate prescription, was alleviated when the compound of chemical formula 1 was administered. The flushing phenomenon was confirmed through analysis of the concentration of prostaglandin D2 (PGD2) in the blood. PGD2 is a bio-derived substance closely related to the flushing phenomenon, and the concentration analysis of PGD2 in the blood is known as a standard test method for predicting the side effects of drugs. (The journal of pharmacology and experimental therapeutics, 2008, 327(3), 665.)

The test method was conducted by dividing C57BL6 male mice into three groups as test animals. (G1: vehicle, G2: dimethyl fumarate 60 mg/kg, G3: compound of chemical formula 1 165.6 mg/kg, n=3) In each test group, the test substance used an excipient of 10% NMP, 90% corn oil, and blood was collected 5 minutes after a single oral administration. PGD2 present in the collected blood was measured using an Elisa kit (#CSB-E14022m, CUSABIO).

The results are shown in FIG. 4. As shown in FIG. 4, 5 minutes after drug administration, the group administered with the compound of chemical formula 1 (32.95±13.1 pg/ml) showed a significant decrease of 76.6% compared to the group administered with dimethyl fumarate (140.74±29.5 pg/ml).

In conclusion, the increase in blood PGD2, a biomarker of flushing side effects induced by dimethyl fumarate, was significantly reduced in the group administered the compound of chemical formula 1, and it was judged that there is a high possibility of clinically alleviating toxicity.

1. A compound represented by the following chemical formula 1.

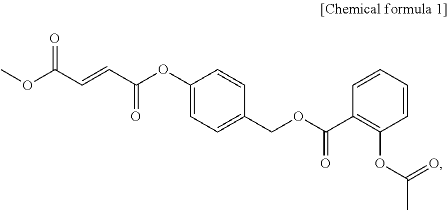 [SMILES: COC(=O)/C=C/C(=O)Oc1ccc(COC(=O)c2ccccc2OC(C)=O)cc1]

2. A pharmaceutical composition comprising the compound of claim 1 and a pharmaceutically acceptable carrier.

3. A method of treating or ameliorating psoriasis, atopy, macular degeneration, multiple sclerosis, asthma, arthritis, inflammatory bowel disease, lupus, amyotrophic lateral sclerosis, Huntington's disease, Alzheimer's disease, Parkinson's disease, sleep apnea, radiologically isolated syndrome, scleroderma, neuropathic pain, chronic pain, gout, diabetic complications including diabetic foot ulcers, cerebrovascular disease, cardiovascular disease, cancer or tumor, comprising administering to a subject in need thereof a therapeutically effective amount of the compound of clam 1.
